(19) United States
(12) Patent Application Publication
(10) Pub. No.: US 2025/0161224 A1
(43) Pub. Date: May 22, 2025

(54) METHOD FOR PREPARING MICROSPHERE WITH IMPROVED SUSPENSION PERFORMANCE, USING COACERVATION

(71) Applicant: DONG KOOK PHARM. CO., LTD., Seoul (KR)

(72) Inventors: Kwang Jong KIM, Gyeonggi-do (KR); Soo Yeon KANG, Seoul (KR); In Hyeok OH, Gyeonggi-do (KR); Byung Hyuck KIM, Gyeonggi-do (KR); Su Min HAN, Gyeonggi-do (KR); Keon II KIM, Gyeonggi-do (KR)

(21) Appl. No.: 18/842,478

(22) PCT Filed: Mar. 7, 2023
(86) PCT No.: PCT/KR2023/003080

§ 371 Date: Aug. 29, 2024

(30) Foreign Application Priority Data
Mar. 8, 2022 (KR) 10-2022-0029607

(51) Int. Cl. A61K 9/50; A61K 38/31

(52) U.S. Cl. CPC A61K 9/5031; A61K 38/31

(57) ABSTRACT

The present invention discloses a method for preparing microspheres with improved suspension performance, using a coacervation method.

### TECHNICAL FIELD

The present invention relates to a method of producing microspheres with improved suspendability using a coacervation method, and more specifically, to a production method capable of significantly improving the suspendability and injectability of microspheres by a washing process using a surfactant.

### BACKGROUND

Biodegradable microspheres are a dosage form developed for the purpose of long-term release of drugs in vivo, and may deliver drugs by subcutaneous injection or intramuscular injection.

Here, low suspendability and injectability of microspheres may reduce patient compliance because the microspheres clog the needle of the syringe upon in vivo injection or need to be injected using a larger syringe.

In general, the injectability of microspheres may be improved by making the particle size of the microspheres uniform or reducing the average particle size thereof.

However, this method has problems in that the physicochemical properties of the microspheres may vary depending on the particle size or distribution and the production method is complicated.

### PRIOR ART DOCUMENTS

### Patent Documents

(Patent Document 1) European Patent Publication No. 0058481
(Patent Document 2) European Patent Publication No. 0092918

### DISCLOSURE

### Technical Problem

An object of the present invention is to provide a method of producing microspheres using a coacervation method, which does not affect the physicochemical properties of drug-containing microspheres, uses a simple process, and may improve the water suspendability and injectability of microspheres.

Specifically, an object of the present invention is to provide a method of producing microspheres with improved suspendability using a coacervation method, and more specifically, an object of the present invention is to provide a method comprising a process of washing with an aqueous solution containing a surfactant.

Furthermore, a specific object of the present invention is to provide the method wherein the process of washing with an aqueous solution containing a surfactant such as polyvinyl alcohol or poloxamer is a continuous process without exposure to an external environment.

In addition, a special object of the present invention is to provide a method of producing microspheres using a coacervation method, which is capable of significantly improving the suspendability of microspheres in a solvent for injection and the injectability thereof by comprising a washing process.

### Technical Solution

To achieve the above objects, the present invention discloses the following means.

In one aspect, the present invention discloses a method of producing microspheres with improved suspendability using a coacervation method, comprising steps of: (S1) preparing a crude solution by adding and dispersing a second solution containing a pharmaceutical compound dissolved therein to a first solution containing a biocompatible polymer carrier substance dissolved therein; (S2) inducing the formation of microspheres by adding a phase inducer to the dispersion obtained in step (S1) to cause phase separation; (S3) hardening the microspheres by adding an emulsion to the mixture obtained in step (S2); (S4) washing the hardened microspheres of step (S3) with an aqueous solution containing a surfactant; (S5) recovering the washed microspheres of step (S4); and (S6) freeze-drying the recovered microspheres of step (S5).

### Advantageous Effects

In the production method according to the present invention, the washing process using a surfactant is a continuous process without exposure to an external environment during the coacervation process and has the advantage of being able to be simply added and used.

In addition, the production method according to the present invention comprises a process of washing with an aqueous solution containing a surfactant after the hardening step, and thus may increase the wettability of the surface of the microspheres, thereby effectively improving the suspendability and injectability of the microspheres, unlike a process without a washing step.

Therefore, when the microspheres produced by the production method according to the present invention are used as an injection agent, they have the effect of improving patient compliance due to a reduced needle size resulting from the improved injectability thereof compared to commercial products.

The effects of the present invention are not limited to the effects mentioned above, and may various effects within a range obvious to those skilled in the art from the following description.

### BRIEF DESCRIPTION OF DRAWINGS

FIG. 1 is a flow chart showing a method of producing microspheres with improved suspendability using a coacervation method according to the present invention.

FIG. 2 depicts photographs showing the results of SEM analysis performed to analyze the morphology of microspheres produced in Examples 2 to 4.

FIG. 3 depicts photographs showing the results of SEM analysis performed to analyze the morphology of microspheres produced in Examples 5 to 7.

FIGS. 4, 5 and 6 depict graphs showing the results of in vitro drug release evaluation of octreotide for microspheres (unwashed formulation) produced in Comparative Example 1, microspheres (formulation washed with PVA) produced in Examples 2 to 4, and microspheres (formulation washed with poloxamer 188) produced in Examples 5 to 7.

FIG. 7 is a graph showing the results of an injection force test for microspheres according to Examples 2 to 4.

FIG. 8 is a graph showing the results of an injection force test for microspheres according to Examples 5 to 7.

FIG. 9 depicts photographs showing the results of an injectability test for microspheres according to Comparative Example 1 and Example 1.

FIG. 10 depicts photographs showing the results of a suspension test for microspheres according to Comparative Example 1 and Example 1.

FIG. 11 is a graph showing the blood concentration profile obtained after administering microspheres produced according to Comparative Example 1 and Example 1 to rats.

### BEST MODE

Hereinafter, the present specification will be described in more detail.

This is explained in detail as follows. The terms used in the present specification are currently widely used general terms selected in consideration of their functions in the present invention, but they may change depending on the intents of those skilled in the art, precedents, or the advents of new technology. Additionally, in certain cases, there may be terms arbitrarily selected by the applicant, and in this case, their meanings are described in a corresponding description part of the present invention. Accordingly, terms used in the present invention should be defined based on the meaning of the term and the entire contents of the present invention, rather than the simple term name.

Unless otherwise defined, all terms used herein, including technical or scientific terms, have the same meanings as understood by those skilled in the art to which the present invention pertains. Terms such as those used in general and defined in dictionaries should be interpreted as having meanings identical to those specified in the context of related technology. Unless definitely defined in the present application, the terms should not be interpreted as having ideal or excessively formative meanings.

A numerical range includes numerical values defined in the range. Every maximum numerical limitation given throughout the present specification includes every lower numerical limitation, as if such lower numerical limitations were expressly written herein. Every minimum numerical limitation given throughout the present specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Furthermore, each description and embodiment disclosed in the present invention may also be applied to other descriptions and embodiments. That is, all combinations of the various components disclosed in the present invention fall within the scope of the present invention. In addition, the scope of the present invention cannot be construed as being limited by the specific descriptions described below.

As used herein, terms such as “containing” or “comprising” should be understood as open-ended terms implying the possibility of including other embodiments.

In order to achieve the above-described objects, the present inventors have conducted research and development on a method of producing drug-encapsulated microspheres using a coacervation method. As a result, the present inventors have found that, when a washing process using an aqueous solution containing a surfactant is additionally performed, it is possible to improve the suspendability and injectability of microspheres by increasing the surface wettability of the microspheres, unlike a process without a washing process, and accordingly, when the microspheres are used as an injection agent, it is possible improve patient compliance due to a reduced needle size resulting from the improved injectability of the microspheres compared to a commercial product (Sandostatin, 19-gauge), thereby completing the present invention.

Hereinafter, the present invention will be described in detail.

Method of Producing Microspheres with Improved Suspendability Using Coacervation Method

To achieve the above-described objects, the present invention discloses the following means.

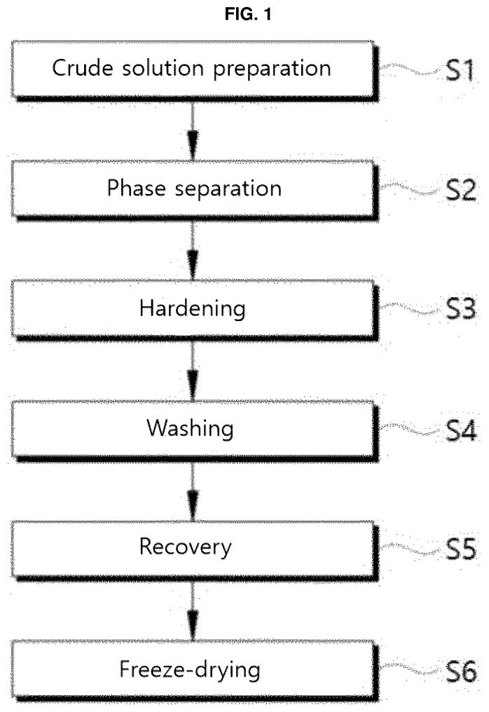

Referring to FIG. 1, in one aspect, the present invention provides a method of producing microspheres with improved suspendability using a coacervation method, comprising steps of: (S1) preparing a crude solution by adding and dispersing a second solution containing a pharmaceutical compound dissolved therein to a first solution containing a biocompatible polymer carrier substance dissolved therein; (S2) inducing the formation of microspheres by adding a phase inducer to the dispersion obtained in step (S1) to cause phase separation; (S3) hardening the microspheres by adding an emulsion to the mixture obtained in step (S2); (S4) washing the hardened microspheres of step (S3) with an aqueous solution containing a surfactant; (S5) recovering the washed microspheres of step (S4); and (S6) freeze-drying the recovered microspheres of step (S5).

In the present invention, the term “coacervation method” refers to a method of producing microspheres by separating a concentrated colloidal sol from a dispersion medium by a decrease in hydration of colloidal particles in a hydrophilic colloidal solution and an electrostatic factor.

In the present invention, the first solution in step (S1) is prepared using a first solvent capable of dissolving only the biocompatible polymer carrier substance without dissolving the pharmaceutical compound, and the first solvent in the first solution may be methylene chloride, without being limited thereto.

In the present invention, the second solution in step (S1) is prepared using a second solvent capable of dissolving only the pharmaceutical compound without dissolving the biocompatible polymer carrier substance, and the second solvent in the second solution may be methanol, without being limited thereto.

In the present invention, the biocompatible polymer carrier substance in step (S1) may be any one or more selected from the group consisting of polylactic acid (PLA), polylactic-co-glycolic acid (PLGA), polyphosphazene, poly(imino carbonate), polyphosphoester, polyanhydride, polyorthoester, a copolymer of lactic acid and caprolactone, polycaprolactone, polyhydroxyvalerate, polyhydroxybutyrate, polyamino acid, a copolymer of lactic acid and amino acid, and mixtures thereof, without being limited thereto.

In the present invention, the pharmaceutical compound in step (S1) may be a peptide drug, wherein the peptide drug may be octreotide or a salt thereof, wherein the salt of octreotide may be octreotide acetate, without being limited thereto.

In the present invention, the phase inducer in step (S2) may be silicone oil, without being limited thereto.

In the present invention, the emulsion in step (S3) may be an oil-in-water (O/W) type emulsion, and may include heptane, phosphate buffer (pH 4), silicone oil, and sorbitan oleate, without being limited thereto.

In the present invention, the surfactant in step (S4) may be a nonionic surfactant, and may be any one or more selected from the group consisting of polysorbate, poloxamer, polyethylene glycol, and polyvinyl alcohol. Specifically, the surfactant may be poloxamer or polyvinyl alcohol, without being limited thereto.

In the present invention, the concentration of the surfactant in step (S4) may be 0.05 to 7 wt % based on the total weight of the aqueous solution. Specifically, the concentration of the surfactant may be 0.1 to 5 wt % based on the total weight of the aqueous solution, without being limited thereto.

Specifically, when a washing process is performed with an aqueous solution containing the surfactant at a concentration within the above range, there are advantages in that it is possible to the suspendability and injectability of drug-encapsulated microspheres, and it is possible to improve the appearance of the produced microspheres by increasing the yield.

In the present invention, the washing process in step (S4) may be a continuous process without exposure to an external environment during a coacervation process, without being limited thereto.

### Microspheres Produced Using Coacervation Method

In one aspect, the present invention provides microspheres produced by the above-described production method.

In the present invention, the average particle size of the microspheres may be 30 to 50 μm, without being limited thereto.

In the present invention, the span value of the microspheres may be 1.55 or less. Specifically, when the microspheres are washed with an aqueous solution containing polyvinyl alcohol, the span value of the microspheres may be 1.10 to 1.34, and when the microspheres are washed with an aqueous solution containing poloxamer, the span value of the microspheres may be 1.14 to 1.55, without being limited thereto.

In the present invention, the pharmaceutical compound contained in the microsphere may be encapsulated in an amount of 10 to 30 mg, without being limited thereto.

In the present invention, the dissolution pattern of the microspheres containing the pharmaceutical compound, measured using a dissolution solution containing acetonitrile at pH 10, may satisfy all of the following conditions, without being limited thereto:
1) the dissolution rate of the pharmaceutical compound at a dissolution time of 1 hour is 10.0%; and
2) the dissolution rate of the pharmaceutical compound at a dissolution time of 24 hours is 60% to 90%.

### Pharmaceutical Composition for Parenteral Administration Comprising Microspheres

In one aspect, the present invention provides a pharmaceutical composition for parenteral administration comprising the microspheres.

In the present invention, the term “carrier” refers to a compound that facilitates the addition of a compound into a cell or tissue, and the term “pharmaceutically acceptable” refers to a composition which is physiologically acceptable and, when administered to humans, does not cause allergic reactions such as gastrointestinal disorders and dizziness, or similar reactions.

In one embodiment of the present invention, the pharmaceutically acceptable carrier is one commonly used for formulation, and examples thereof include, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil.

In the present invention, the term “parenteral administration” refers to administration by injection, excluding oral administration. Specifically, the route of administration by injection may be any of subcutaneous injection, intradermal injection, or intramuscular injection, and the dosage form may be appropriately selected depending on the intended use of the composition.

In addition, the pharmaceutical composition for parenteral administration according to the present invention relates to a pharmaceutical composition for the prevention or treatment of acromegaly, gastrointestinal and pancreatic endocrine tumors, or progressive neuroendocrine tumors, and may reduce the needle size (23- to 26-gauge) compared to a conventional commercially available product (Sandostatin, 19-gauge), thereby improving patient compliance.

In the present invention, the term “prevention” means delaying the onset of acromegaly, gastrointestinal and pancreatic endocrine tumors, or progressive neuroendocrine tumors. If the onset of acromegaly, gastrointestinal and pancreatic endocrine tumors, or progressive neuroendocrine tumors is delayed for an expected period of time, prevention may be considered complete.

In the present invention, the term “treatment” means partially or completely mitigating, ameliorating, alleviating, inhibiting or delaying the onset of acromegaly, gastrointestinal or pancreatic endocrine tumors, or progressive neuroendocrine tumors, thereby reducing the severity or reducing incidence of one or more symptoms or features.

The matters mentioned with respect to the production method of the present invention, the microspheres produced thereby, and the parenteral pharmaceutical composition comprising the same are applied equally unless they are mutually contradictory.

Hereinafter, the present invention will be described in detail by way of examples. However, the following examples are only intended to illustrate the present invention, and the scope of the present invention is not limited thereto.

### Examples

### Example 1. Production of Octreotide Acetate-Encapsulated Microspheres Using Coacervation Method (Washing Process Using Polyvinyl Alcohol)

About 3.8 g of a linear or branched polymer (50/50 mol, M_{w}=47,000) consisting of poly(D,L-lactide-glycolide) was dissolved in 43 mL of methylene chloride, and then 224.6 mg of octreotide acetate (200.5 mg as octreotide) dissolved in about 0.9 mL of methanol was added to the solution, thereby preparing a peptide-polymer mixed solvent. About 60 mL of silicone oil (polydimethylsiloxane, Dow 360 Medical Fluid, 1000 cs) was added to the peptide-polymer mixed solvent at an appropriate rate. The resulting mixture was added to a stirred emulsion containing 1,200 mL of n-heptane, 270 mL of phosphate buffer (pH 4), 35 mL of silicone oil (polydimethylsiloxane, Dow 360 Medical Fluid, 350 cs), and 2 mL of the emulsifier sorbitan oleate (Span 80®). Microspheres were produced by continued stirring at an appropriate stirring speed for a predetermined period of time.

The produced microspheres were recovered by vacuum filtration, and then stirred for at least 30 minutes in an aqueous solution containing 0.5 wt % (w/w) of polyvinyl alcohol (PVA, Sigma-Aldrich, Lot.no: P8136, product name: Poly(vinyl alcohol) 87-90% hydrolyzed, average mol wt 30,000-70,000) (the concentration of PVA is 0.5 wt % based on the total weight of the aqueous solution) dissolved therein and recovered. The recovered microspheres were freeze-dried overnight and then obtained (yield: 71.3%, average particle size: 31.4 μm).

### Example 2. Production of Octreotide Acetate-Encapsulated Microspheres Using Coacervation Method (Washing Process Using Polyvinyl Alcohol)

Octreotide acetate-encapsulated microspheres were produced under the same conditions as in Example 1 (yield: 63.5%, average particle size: 34.1 μm), except that an “aqueous solution containing 0.1 wt % of polyvinyl alcohol (PVA) (the concentration of PVA is 0.1 wt % based on the total weight of the aqueous solution)” dissolved therein was used instead of the “aqueous solution containing 0.5 wt % of polyvinyl alcohol (PVA) dissolved therein”.

### Example 3. Production of Octreotide Acetate-Encapsulated Microspheres Using Coacervation Method (Washing Process Using Polyvinyl Alcohol)

Octreotide acetate-encapsulated microspheres were produced under the same conditions as in Example 1 (yield: 60.4%, average particle size: 32.5 μm), except that an “aqueous solution containing 1.0 wt % of polyvinyl alcohol (PVA) (the concentration of PVA is 1.0 wt % based on the total weight of the aqueous solution)” dissolved therein was used instead of the “aqueous solution containing 0.5 wt % of polyvinyl alcohol (PVA) dissolved therein”.

### Example 4. Production of Octreotide Acetate-Encapsulated Microspheres Using Coacervation Method (Washing Process Using Polyvinyl Alcohol)

Octreotide acetate-encapsulated microspheres were produced under the same conditions as in Example 1 (yield: 57.1%, average particle size: 30.8 μm), except that an “aqueous solution containing 5.0 wt % of polyvinyl alcohol (PVA) (the concentration of PVA is 5.0 wt % based on the total weight of the aqueous solution)” dissolved therein was used instead of the “aqueous solution containing 0.5 wt % of polyvinyl alcohol (PVA) dissolved therein”.

### Example 5. Production of Octreotide Acetate-Encapsulated

Microspheres Using Coacervation Method (Washing Process Using Poloxamer 188 (P188)))

About 3.8 g of a linear or branched polymer (50/50 mol, M_{w}=47,000) consisting of poly(D,L-lactide-glycolide) was dissolved in 43 mL of methylene chloride, and then 224.6 mg of octreotide acetate (200.5 mg as octreotide) dissolved in about 0.9 mL of methanol was added to the solution, thereby preparing a peptide-polymer mixed solvent. About 60 mL of silicone oil (Dow 360 Medical Fluid, 1000 cs) was added to the peptide-polymer mixed solvent at an appropriate rate. The resulting mixture was added to a stirred emulsion containing 1,200 mL of n-heptane, 270 mL of phosphate buffer (pH 4), 35 mL of silicone oil (polydimethylsiloxane, Dow 360 Medical Fluid, 350 cs), and 2 mL of the emulsifier sorbitan oleate (Span 80©). Microspheres were produced by continued stirring at an appropriate stirring speed for a predetermined period of time.

The produced microspheres were recovered by vacuum filtration, and then stirred for at least 30 minutes in an aqueous solution containing 0.1 wt % (w/w) of Poloxamer 188 (average molecular weight: 8,905 g/mol) (the concentration of Poloxamer 188 is 0.1 wt % based on the total weight of the aqueous solution) dissolved therein and recovered. The recovered microspheres were freeze-dried overnight and then obtained (yield: 54.3%, average particle size: 34.2 μm).

### Example 6. Production of Octreotide Acetate-Encapsulated Microspheres Using Coacervation Method (Washing Process Using Poloxamer 188 (P188)))

Octreotide acetate-encapsulated microspheres were produced under the same conditions as in Example 5 (yield: 61.7%, average particle size: 32.2 μm), except that an “aqueous solution containing 1.0 wt % of Poloxamer 188 (the concentration of Poloxamer 188 is 1.0 wt % based on the total weight of the aqueous solution)” dissolved therein was used instead of the “aqueous solution containing 0.1 wt % of Poloxamer 188 dissolved therein”.

### Example 7. Production of Octreotide Acetate-Encapsulated Microspheres Using Coacervation Method (Washing Process Using Poloxamer 188 (P188)))

Octreotide acetate-encapsulated microspheres were produced under the same conditions as in Example 5 (yield: 63.3%, average particle size: 33.8 μm), except that an “aqueous solution containing 5.0 wt % of Poloxamer 188 (the concentration of Poloxamer 188 is 5.0 wt % based on the total weight of the aqueous solution)” dissolved therein was used instead of the “aqueous solution containing 0.1 wt % of Poloxamer 188 dissolved therein”.

### Comparative Example 1. Production of Octreotide Acetate-Encapsulated Microspheres Using Coacervation Method (without Washing Process)

About 3.8 g of a linear or branched polymer (50/50 mol, M_{w}=47,000) consisting of poly(D,L-lactide-glycolide) was dissolved in 43 mL of methylene chloride, and then 224.6 mg of octreotide acetate (200.5 mg as octreotide) dissolved in about 0.9 mL of methanol was added to the solution, thereby preparing a peptide-polymer mixed solvent. About 60 mL of silicone oil (Dow 360 Medical Fluid, 1000 cs) was added to the peptide-polymer mixed solvent at an appropriate rate. The resulting mixture was added to a stirred emulsion containing 1,200 mL of n-heptane, 270 mL of phosphate buffer (pH 4), 35 mL of silicone oil (polydimethylsiloxane, Dow 360 Medical Fluid, 350 cs), and 2 mL of the emulsifier sorbitan oleate (Span 80©). Microspheres were produced by continued stirring at an appropriate stirring speed for a predetermined period of time.

The produced microspheres were recovered by vacuum filtration and then freeze-dried overnight and then obtained (yield: 30.5%, average particle size: 49.0 μm).

### Comparative Example 2. Production of Octreotide Acetate-Encapsulated Microspheres Using Coacervation Method (Washing Process Using Distilled Water)

About 3.8 g of a linear or branched polymer (50/50 mol, M_{w}=47,000) consisting of poly(D,L-lactide-glycolide) was dissolved in 43 mL of methylene chloride, and then 224.6 mg of octreotide acetate (200.5 mg as octreotide) dissolved in about 0.9 mL of methanol was added to the solution, thereby preparing a peptide-polymer mixed solvent. About 60 mL of silicone oil (polydimethylsiloxane, Dow 360 Medical Fluid, 1000 cs) was added to the peptide-polymer mixed solvent at an appropriate rate. The resulting mixture was added to a stirred emulsion containing 1,200 mL of n-heptane, 270 mL of phosphate buffer (pH 4), 35 mL of silicone oil (polydimethylsiloxane, Dow 360 Medical Fluid, 350 cs), and 2 mL of the emulsifier sorbitan oleate (Span 80©). Microspheres were produced by continued stirring at an appropriate stirring speed for a predetermined period of time.

The produced microspheres were recovered by vacuum filtration, and then stirred for at least 30 minutes in 1,200 mL of distilled water and recovered. The recovered microspheres were freeze-dried overnight and then obtained (yield: 40.7%, average particle size: 33.6 μm).

### Experimental Examples

### Experimental Example 1. Microsphere Size Measurement and Morphology Evaluation

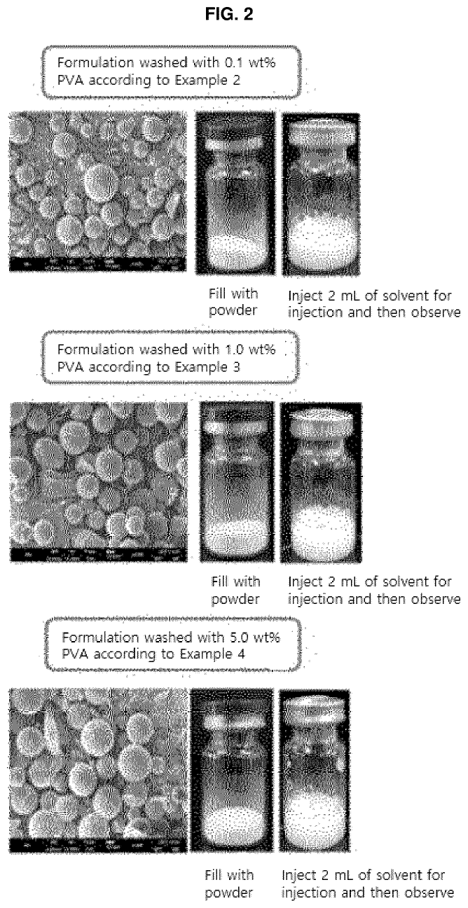

The particle size distributions of the microspheres produced in Examples 2 to 4 were analyzed using a particle size analyzer (Malvern-MASTERSLZER 3000), and the results are shown in Table 1 below. In addition, the morphology of the microspheres was analyzed using a scanning electron microscope (SEM, EmCRAFTS-CUBE II), and the results are shown in FIG. 2.

Referring to Table 1, the d₁₀, d₅₀, and d₉₀ of the microspheres produced according to Example 2 were measured as 19.8, 34.1, and 63.2 μm, respectively, the d₁₀, d₅₀, and d₉₀ of the microspheres produced according to Example 3 were measured as 18.6, 32.5, and 62.0 μm, respectively, and the d₁₀, d₅₀, and d₉₀ of the microspheres produced according to Example 4 were measured as 18.4, 30.8, and 52.2 μm, respectively. The span (meaning the width of particle size distribution) values calculated from these results using Equation 1 below were 1.28 (Example 2), 1.34 (Example 3), and 1.10 (Example 4).

$$Span = {d}_{90} - {d}_{10} / {d}_{50} \qquad [ Equation 1 ]$$

The span values were measured to be less than 2.0, suggesting that the microspheres produced according to Examples 2 to 4 have a uniform size. In addition, it was shown that the microspheres produced according to Examples 2 to 4 have a size that can be administered in vivo by intramuscular or subcutaneous injection.

In addition, referring to FIG. 2, it was confirmed that, as the microspheres produced in Examples 2 to 4 were subjected to the process of washing with the aqueous solution containing polyvinyl alcohol, the microspheres did not aggregate and had a uniform appearance.

From the above results, it could be confirmed that there was no significant difference in the physicochemical evaluation (particle size) between the microspheres according to Examples 2 to 4, suggesting that the above-described concentration range of polyvinyl alcohol (0.1 to 5%) is a range suitable for pharmaceutical quality.

TABLE 1

| | Formulation washed | Formulation washed | Formulation washed |
|---|---|---|---|
| | with 0.1% PVA | with 1.0% PVA | with 5.0% PVA |
| | according to | according to | according to |
| | Example 2 | Example 3 | Example 4 |
| Particle size | 19.8/34.1/63.2 | 18.6/32.5/62.0 | 18.4/30.8/52.2 |
| (μm) | | | |
| d₁₀/d₅₀/d₉₀ | | | |

(In Table 1 above, d₁₀, d₅₀ and d₉₀, which represent the sizes of the microspheres, mean particle sizes corresponding to 10%, 50% and 90% of the cumulative particle distribution, respectively.)

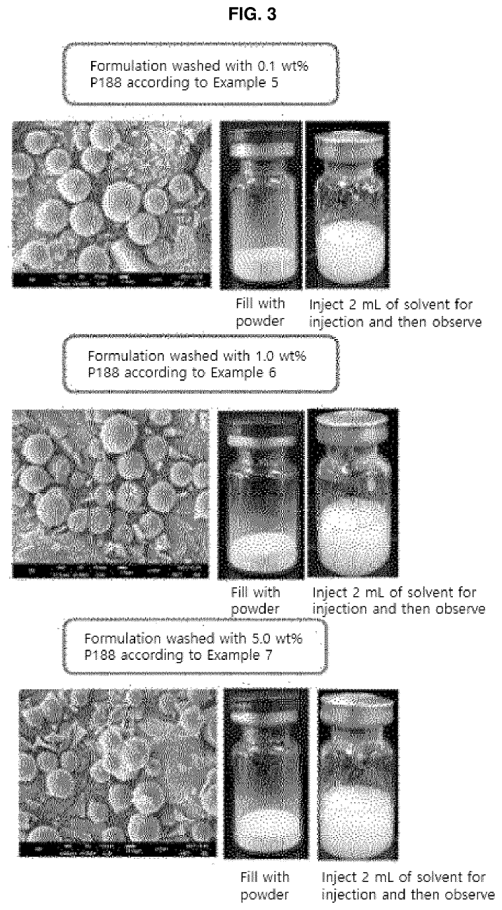

In addition, the particle size distributions and morphologies of the microspheres produced in Examples 5 to 7 were analyzed using the above-mentioned methods, and the results are shown in Table 2 below and FIG. 3.

Referring to Table 2 below, the d₁₀, d₅₀, and d₉₀ of the microspheres produced according to Example 5 were measured as 18.9, 34.2, and 71.8 μm, respectively, the d₁₀, d₅₀, and d₉₀ of the microspheres produced according to Example 6 were measured as 19.1, 32.2, and 55.8 μm, respectively, and the d₁₀, d₅₀, and d₉₀ of the microspheres produced according to Example 7 were measured as 20.0, 33.8, and 58.8 μm, respectively. The span (meaning the width of particle size distribution) values calculated using Equation 1 above were 1.55 (Example 5), 1.14 (Example 6), and 1.15 (Example 7).

The span values were measured to be less than 2.0, suggesting that the microspheres produced according to Examples 5 to 7 have a uniform size. In addition, it was shown that the microspheres produced according to Examples 5 to 7 have a size that can be administered in vivo by intramuscular or subcutaneous injection.

In addition, referring to FIG. 3, it was confirmed that, as the microspheres produced in Examples 5 to 7 were subjected to the process of washing with the aqueous solution containing Poloxamer 188, the microspheres did not aggregate and had a uniform appearance.

From the above results, it could be confirmed that there was no significant difference in the physicochemical evaluation (particle size) between the microspheres according to Examples 5 to 7, suggesting that the above-described concentration range of Poloxamer 188 (0.1 to 5%) is a range suitable for pharmaceutical quality.

TABLE 2

| | Formulation washed | Formulation washed | Formulation washed |
|---|---|---|---|
| | with 0.1% Poloxamer | with 1.0% Poloxamer | with 5.0% Poloxamer |
| | 188 according to | 188 according to | 188 according to |
| | Example 5 | Example 6 | Example 7 |
| Particle size | 18.9/34.2/71.8 | 19.1/32.2/55.8 | 20.0/33.8/58.8 |
| (μm) | | | |
| d₁₀/d₅₀/d₉₀ | | | |

(In Table 2 above, d₁₀, d₅₀ and d₉₀, which represent the sizes of the microspheres, mean particle sizes corresponding to 10%, 50% and 90% of the cumulative particle distribution, respectively.)

### Experimental Example 2. Evaluation of Octreotide Content in Microspheres

The octreotide content in the microspheres produced in each of Examples 2 to 4 was evaluated using a high-performance liquid chromatography (HPLC) analysis method.

Specifically, about 60 mg of the microspheres produced in each of Examples 2 to 4 were placed in a 50 mL flask and completely dissolved in tetrahydrofuran (THF). Then, acetate buffer solution was added up to the marked line and the dissolved octreotide was extracted. Using the extract as the sample, the content of octreotide encapsulated in the microspheres (drug encapsulation rate) was measured using HPLC (instrument name: 1260 infinity LC, Agilent). The results are shown in Table 3 below.

Referring to Table 3, it could be confirmed that the microspheres according to Examples 2 to 4 had a suitable octreotide content and there was no significant difference in the physicochemical evaluation (content) between the microspheres according to Examples 2 to 4, suggesting that the above-described concentration range of polyvinyl alcohol (0.1 to 5%) is a range suitable for pharmaceutical quality.

TABLE 3

| | Formulation washed | Formulation washed | Formulation washed |
|---|---|---|---|
| | with 0.1% PVA | with 1.0% PVA | with 5.0% PVA |
| | according to | according to Example | according to Example |
| | Example 2 | 3 | 4 |
| Content (%) | 102.67% | 102.85% | 107.06% |

In addition, the content of octreotide in the microspheres produced in each of Examples 5 to 7 was analyzed using the method mentioned above, and the results are shown in Table 4 below.

Referring to Table 4, it could be confirmed that the microspheres according to Examples 5 to 7 had a suitable octreotide content and there was no significant difference in the physicochemical evaluation (content) between the microspheres according to Examples 5 to 7, suggesting that the above-described concentration range of Poloxamer 188 (0.1 to 5%) is a range suitable for pharmaceutical quality.

TABLE 4

| | Formulation washed | Formulation washed | Formulation washed |
|---|---|---|---|
| | with 0.1% Poloxamer | with 1.0% Poloxamer | with 5.0% Poloxamer |
| | 188 according to | 188 according to | 188 according to |
| | Example 5 | Example 6 | Example 7 |
| Content (%) | 97.97% | 98.10% | 102.93% |

### Experimental Example 3. In Vitro Drug Release Evaluation of Octreotide

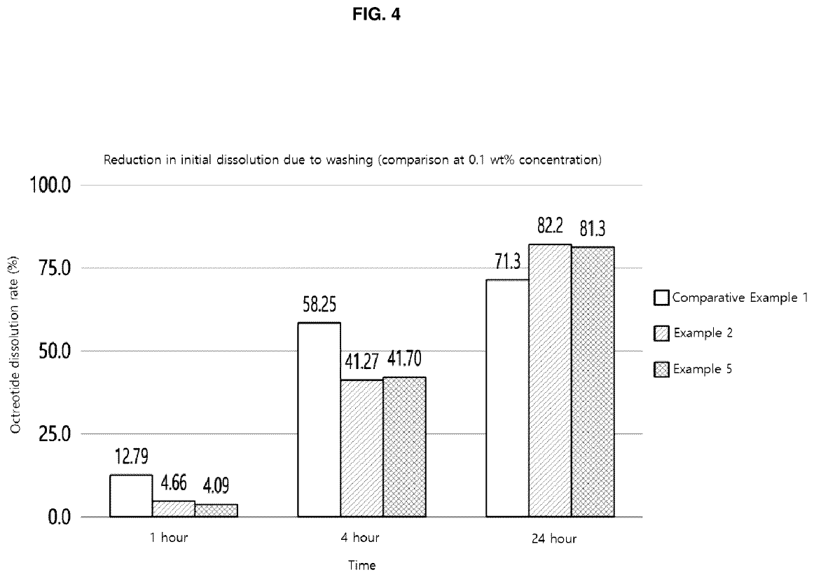

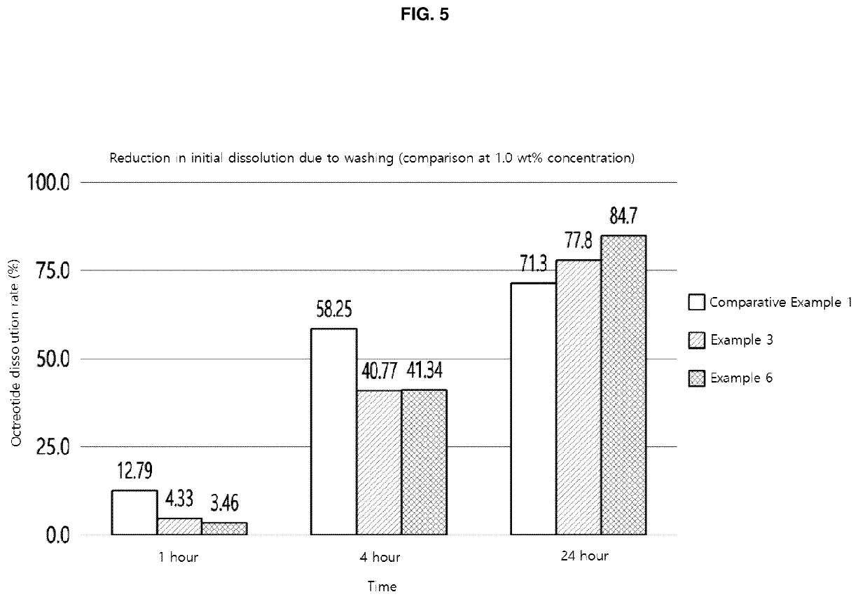

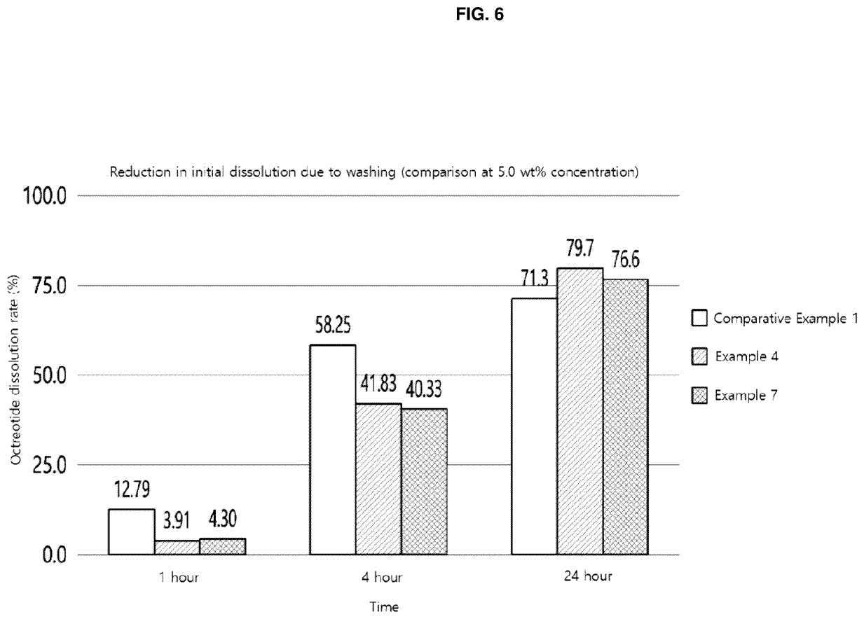

An in vitro release test was conducted for the microspheres (unwashed formulation) produced in Comparative Example 1, the microspheres (formulation washed with PVA) produced in Examples 2 to 4, and the microspheres (formulation washed with Poloxamer 188) produced in Examples 5 to 7. Each 30 mg of the freeze-dried microspheres were dispersed in an elution buffer (pH 10.0) and then subjected to a release test at a constant stirring speed at 37±0.5° C. After sampling for each test time, the obtained microspheres were completely dissolved in tetrahydrofuran (THF), extracted with acetate buffer, and then analyzed by an HPLC analysis method. Here, as the analysis conditions, tetramethylammonium hydroxide/acetylcholine/distilled water (TMAH/ACH/DW) was used as the mobile phase, measurement was performed at 210 nm and a flow rate of 1.8 mL/min, and the dissolution rates at 1 hour, 4 hours, and 24 hours were evaluated. The results are shown in FIGS. 4 to 6.

Referring to FIGS. 4 to 6, it was confirmed that the microspheres (unwashed formulation) according to Comparative Example 1 satisfied the long-term dissolution standard (24-hour standard: dissolution of 58 to 90%), but exceeded the initial dissolution standard (1-hour standard: dissolution of less than 10%, 4-hour standard: dissolution of 10 to 50%).

However, in the case of the microspheres (formulation washed with PVA) according to Examples 2 to 4 and the microspheres (formulation washed with Poloxamer 188) according to Examples 5 to 7, it could be confirmed that the initial dissolution could be suppressed and the long-term dissolution was also satisfied, because the washing process with the aqueous solution containing the surfactant was performed.

### Experimental Example 4. Injection Force Test

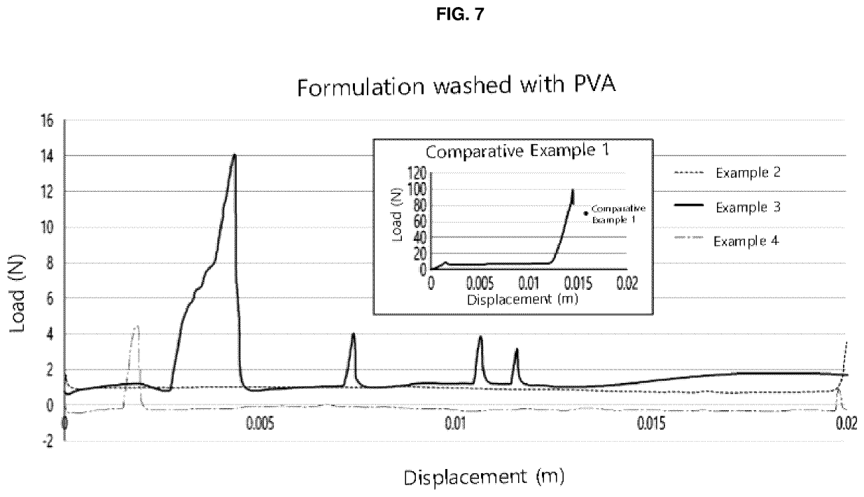

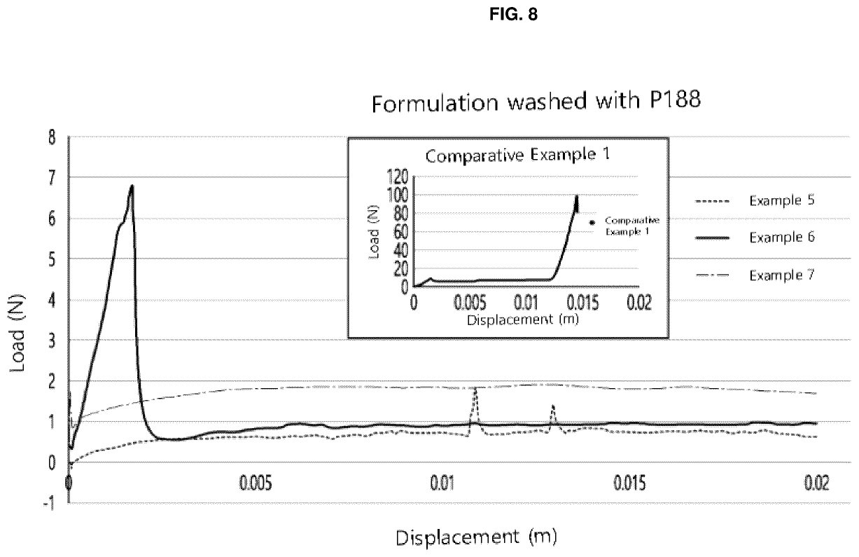

Each quantitative sample was suspended in a solvent for injection solvent through a syringe and 22-gauge and subjected to an injection force test, and the results are shown in FIGS. 7 and 8.

### <Sample Information>

1) Unwashed formulation (Comparative Example 1)
2) Formulation washed with 0.1 wt % PVA (Example 2), formulation washed with 1.0 wt % PVA (Example 3), and formulation washed with 5.0 wt % PVA (Example 4)
3) Formulation washed with 0.1 wt % P188 (Example 5), formulation washed with 1.0 wt % P188 (Example 6), and formulation washed with 5.0 wt % P188 (Example 7)

### (1) Visual Results

It was confirmed that the formulations washed with PVA according to Examples 2 to 4 had good suspendability without the occurrence of bubbles in the solvent for injection within the vial. When injected into the syringe, the formulation washed with PVA was completely transferred into the syringe without any microspheres sticking to the vial wall.

### (2) Results of Injection Force Test

Referring to FIGS. 7 and 8, it was confirmed that the formulation washed with PVA (Examples 2 to 4) and the formulations washed with Poloxamer 188 (Examples 5 to 7) had improved injectability compared to the unwashed formulation (Comparative Example 1).

In addition, it was confirmed that, in the case of the formulation washed with 1.0 wt % PVA, pressure was applied initially, but the overall injection pressure was significantly lower than that of the unwashed formulation.

### Experimental Example 5. Injectability Test

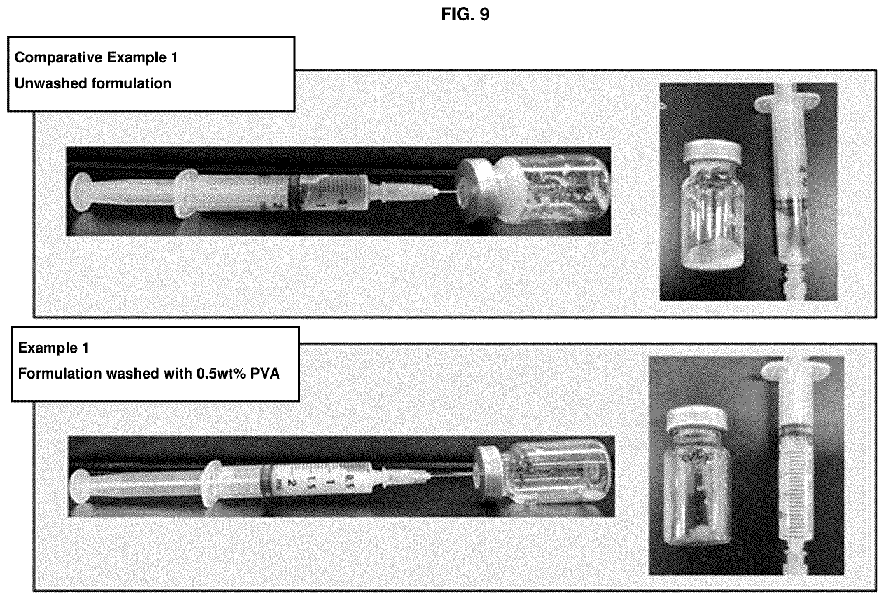

The microspheres (unwashed formulation) according to Comparative Example 1 and the microspheres (formulation washed with 0.5 wt % PVA) according to Example 1 were suspended in a solvent for injection using a 23-gauge 2 mL syringe and subjected to an injectability test, and the results are shown in FIG. 9.

Referring to FIG. 9, it was confirmed that the microspheres (unwashed formulation) according to Comparative Example 1 remained separated from the solvent for injection, and only the solvent for injection existed in the syringe.

However, it was confirmed that the microspheres (formulation washed with 0.5 wt % PVA) according to Example 1 were well suspended in the solvent for injection and were easily introduced into the syringe, suggesting that the injectability was significantly improved by performing the washing process with the aqueous solution containing PVA according to the Example.

### Experimental Example 6. Suspension Test

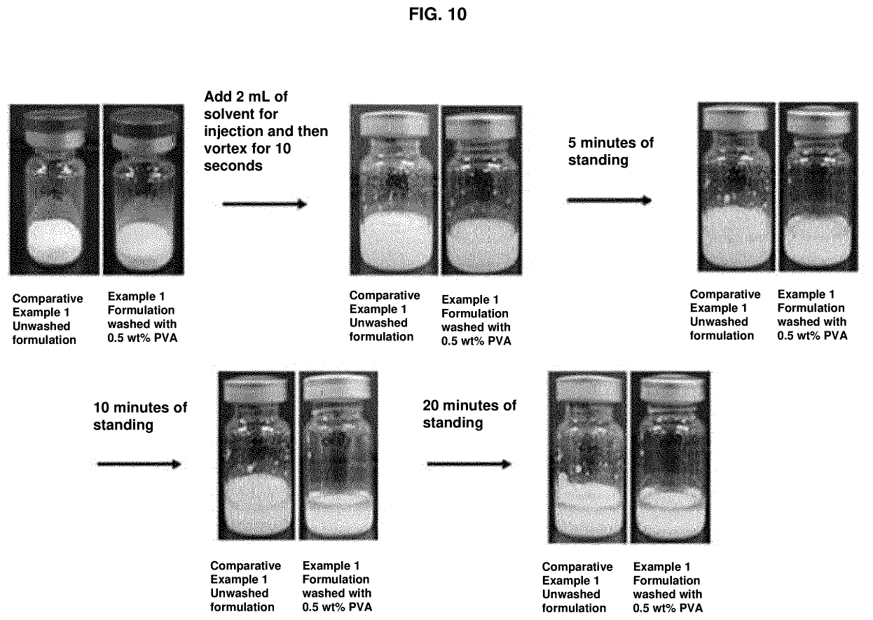

About 625 mg of the microspheres (unwashed formulation) according to Comparative Example 1 and about 625 mg of the microspheres (formulation washed with 0.5 wt % PVA) according to Example 1 were each added to 2 mL of the solvent for injection, and then vortexed for 10 seconds. Thereafter, the appearance of each sample was observed while standing (standing times: 5 minutes, 10 minutes, and 20 minutes), and the results are shown in FIG. 10.

Referring to FIG. 10, it was confirmed that, in the case of the microspheres (unwashed formulation) according to Comparative Example 1, foaming occurred after 5 minutes of standing, and the appearance was bulky.

However, it was confirmed that, in the case of the microspheres (formulation washed with 0.5 wt % PVA) according to Example 1, no foaming occurred and the appearance was soft.

From the above results, it can be seen that the microspheres (formulation washed with 0.5 wt % PVA) according to Example 1 can be administered in a predetermined amount without foaming in the syringe.

### Experimental Example 7. Individual Variation in Blood Octreotide Concentration

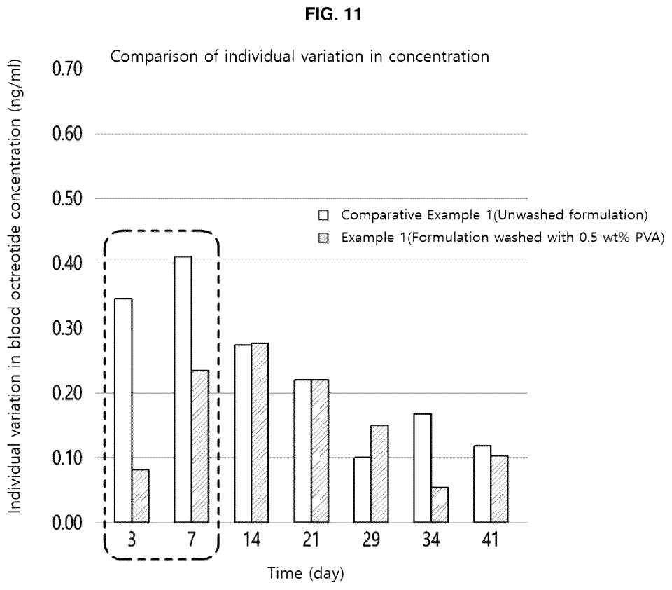

About 53 mg of the microspheres (unwashed formulation) according to Comparative Example 1 and about 53 mg of the microspheres (formulation washed with 0.5 wt % PVA) according to Example 1 were suspended in a vehicle and administered intramuscularly (I.M.) to rats at an octreotide dose of 10 mg/kg. About 0.5 mL of a blood sample was collected at predetermined time points, pretreated, and then analyzed for the blood octreotide concentration using LC/MS/MS. The results are shown in Table 5 below and FIG. 11.

Referring to Table 5 and FIG. 11, it was confirmed that, when the formulation washed with PVA was administered, individual variation was not improved at all the blood collection time points, but individual variation was reduced at specific time points (day 3 and day 7).

TABLE 5

| In vivo | | |
|---|---|---|
| (time, day) | Comparative Example 1 | Example 1 |
| 3 | 0.35 | 0.08 |
| 7 | 0.41 | 0.24 |
| 14 | 0.28 | 0.28 |
| 21 | 0.22 | 0.22 |
| 29 | 0.10 | 0.15 |
| 34 | 0.17 | 0.06 |
| 41 | 0.12 | 0.10 |

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

What is claimed is:

1. A method of producing microspheres with improved suspendability using a coacervation method, comprising steps of:
(S1) preparing a crude solution by adding and dispersing a second solution containing a pharmaceutical compound dissolved therein to a first solution containing a biocompatible polymer carrier substance dissolved therein;
(S2) inducing formation of microspheres by adding a phase inducer to the dispersion obtained in step (S1) to cause phase separation;
(S3) hardening the microspheres by adding an emulsion to the mixture obtained in step (S2);
(S4) washing the hardened microspheres of step (S3) with an aqueous solution containing a surfactant;
(S5) recovering the washed microspheres of step (S4); and
(S6) freeze-drying the recovered microspheres of step (S5).

2. The method of claim 1, wherein the biocompatible polymer carrier substance is any one or more selected from the group consisting of polylactic acid (PLA), polylactic-co-glycolic acid (PLGA), polyphosphazene, poly(imino carbonate), polyphosphoester, polyanhydride, polyorthoester, a copolymer of lactic acid and caprolactone, polycaprolactone, polyhydroxyvalerate, polyhydroxybutyrate, polyamino acid, a copolymer of lactic acid and amino acid, and mixtures thereof.

3. The method of claim 1, wherein the pharmaceutical compound is a peptide drug.

4. The method of claim 3, wherein the peptide drug is octreotide or a salt thereof.

5. The method of claim 1, wherein the surfactant is any one or more selected from the group consisting of polysorbate, poloxamer, polyethylene glycol, and polyvinyl alcohol.

6. The method of claim 1, wherein a concentration of the surfactant is 0.05 to 7 wt % based on the total weight of the aqueous solution.
